(19) United States
(12) Patent Application Publication
(10) Pub. No.: US 2011/0269633 A1
(43) Pub. Date: Nov. 3, 2011

(54) INFLAMMATORY BIOMARKERS FOR MONITORING DEPRESSIVE DISORDERS

(76) Inventors: John Bilello, Durham, NC (US); Yiwu He, Mercer Island, WA (US); Bo Pi, Carlsbad, CA (US)

(21) Appl. No.: 12/922,365

(22) PCT Filed: Mar. 11, 2009
(86) PCT No.: PCT/US09/36833

§ 371 Date: May 12, 2011

Related U.S. Application Data
(60) Provisional application No. 61/036,013, filed on Mar. 12, 2008.

(51) Int. Cl. C40B 30/02; C12Q 1/02; G06F 19/10; C40B 30/04

(52) U.S. Cl. 506/8; 506/9; 435/29; 702/19

(57) ABSTRACT

Materials and Methods related to developing a unipolar depression (MDD) disease score in a subject using a multi-parameter system to measure a plurality of parameters, and an algorithm to calculate a score.

### CROSS-REFERENCE TO RELATED APPLICATIONS

This patent document claims benefit of priority from U.S. Provisional Application Ser. No. 61/036,013, entitled “Inflammatory Biomarkers for Monitoring Depression Disorders,” filed on Mar. 12, 2008, which is incorporated herein by reference in its entirety.

### TECHNICAL FIELD

This patent document relates to biomarkers and methods for diagnosing and monitoring treatment of medical conditions such as major depressive disorder (MDD).

### BACKGROUND

Neuropsychiatric conditions account for more “years lived with disability” (YLDs) than any other type of clinical condition, accounting for almost 30% of total YLDs (Murray and Lopez (1996) Global Health Statistics: A Compendium of Incidence, Prevalence and Mortality Estimates for over 2000 Conditions Cambridge: Harvard School of Public Health). Unipolar MDD alone accounts for 11% of global YLDs. A number of factors may contribute to sustained disability and less than optimal treatment outcomes, including inaccurate diagnosis, early discontinuation of treatment, social stigma, inadequate antidepressant dosing, antidepressant side effects, and non-adherence to treatment.

Most clinical disorders, including neuropsychiatric condition such as depression disorders, result from interactions between multiple factors rather than from a single biological change. Different individuals affected by the same clinical condition may present with different ranges or extents of symptoms, depending on the specific changes within each individual. The ability to determine depression disease status on an individual basis would be useful for accurate assessment of a subject's specific status. There is a need, however, for reliable methods for diagnosing and determining predisposition to clinical conditions such as depression, and for assessing disease status or response to treatment.

### SUMMARY

This document relates to materials and methods for diagnosing and assessing treatment of depression disorders, including MDD. Clinical assessments and patient interviews are commonly used for diagnosing and monitoring treatment of patients with depression. As described herein, a test based on physiological changes, assessed by measuring biomarkers and deriving a disease score using a computational algorithm, will facilitate earlier treatment of depression and increase acceptance by patients. The techniques described herein can be configured to optimize therapy based on physiological measurements in place of or in addition to clinical assessments and patient interviews.

Biomarkers can provide independent diagnostic or prognostic value by reflecting an underlying condition or disease state. The use of biomarkers can allow for accuracy, reliability, sensitivity, specificity, and predictability for assessing disease status. For example, CRP (C-reactive protein) can be used as a plasma biomarker of low grade systemic inflammation, which can be linked to diverse disorders such as rheumatoid and osteoarthritis, allergies, asthma, Alzheimer's disease, cancer, diabetes, digestive disorders, heart disease, hormonal imbalances, and osteoporosis. While biological markers of inflammation may be useful in monitoring the severity of a specific disease, their clinical utility, particularly in the context of an individual marker, seems limited. It appears, however, that the pattern of inflammatory biomarker expression may differ in different disease syndromes, and the levels of multiple markers may be useful in assessing the severity of disease.

Preliminary studies indicated the value of using multiplexed antibody arrays to develop a panel of biomarkers in populations with MDD. The availability of biological markers reflecting psychiatric state (e.g., the type of pathology, severity, likelihood of positive response to treatment, and vulnerability to relapse) is likely to impact both the appropriate diagnosis and treatment of depression. The systematic, highly parallel, combinatorial approach to assemble “disease specific signatures” using algorithms as described herein can be used to determine the status of MDD, and also to predict an individual's response to therapy.

The examples described in this application are based in part on the identification of methods for diagnosing and monitoring treatment and/or progression of depressive disorders. The methods described herein can include developing an algorithm that includes multiple parameters such as inflammatory biomarkers, measuring the multiple parameters, and using the algorithm to determine a quantitative diagnostic score. In some embodiments, algorithms for application of multiple biomarkers from biological samples such as serum or plasma can be developed for patient stratification, identification of pharmacodynamic markers, and monitoring the efficacy of treatment. Such methods can be used, for example, to monitor the effectiveness of therapy in a depressed individual at an early stage of psychotherapy, cognitive therapy, or antidepressant administration. The methods can include determining whether there has been a change in the plasma biomarkers in an individual treated for depression. Materials and methods are described for developing a unipolar depression (MDD) disease score in a subject, using a multi-parameter system to measure a plurality of parameters and an algorithm to calculate a score. The score determined at two or more time points can be used to determine the progression of MDD or to assess a subject's response to a therapeutic regimen, for example.

The approach described herein differs from some of the more traditional approaches to application of biomarkers, in that a multiple analyte algorithm is used rather than a single marker or a group of single markers. Algorithms can be used to derive a single value that reflects disease status, prognosis, and/or response to treatment. As described herein, highly multiplexed microarray-based immunological tools can be used to simultaneously measure multiple parameters. An advantage of using such tools is that all results can be derived from the same sample and run under the same conditions at the same time. High-level pattern recognition approaches can be applied, and a number of tools are available, including clustering approaches such as hierarchical clustering, self-organizing maps, and supervised classification algorithms (e.g., support vector machines, k-nearest neighbors, and neural networks). The latter group of analytical approaches is likely to be of substantial clinical use.

A basic method can include providing a biological sample (e.g., a blood sample) from a depressed individual; measuring the levels of a group of analytes in the sample; and using an algorithm to determine a MDD disease score. In some embodiments, the method can further include repeating the test after a period of time (e.g., weeks or months); calculating a post-treatment MDD disease score; and comparing the post-treatment score to the earlier score, and also to a control MDD disease score (e.g., an average MDD score determined in normal subjects who do not have a depression disorder). Evidence of a change in a depressed individual's MDD disease score toward a normal value can indicate effectiveness of the therapy. Depending on the nature of the therapeutic regimen, such changes may be observable within the first two months of treatment (e.g., for psychotherapy), or in as little as seven to fourteen days (e.g., for administration of antidepressant therapy).

In one aspect, this document features a method for characterizing depression in a subject, comprising (a) providing numerical values for a plurality of parameters predetermined to be relevant to depression; (b) individually weighting each of said numerical values by a predetermined function, each function being specific to each parameter; (c) determining the sum of the weighted values; (d) determining the difference between said sum and a control value; and (e) if said difference is greater than a predetermined threshold, classifying said subject as having depression, or, if said difference is not different than said predetermined threshold, classifying said subject as not having depression. The depression can be associated with major depressive disorder (MDD).

The parameters can be selected from the group consisting of interleukin-1 (IL-1), interleukin-6 (IL-6), interleukin-7 (IL-7), interleukin-10 (IL-10), interleukin-13 (IL-13), interleukin-15 (IL-15), interleukin-18 (IL-18), alpha-2-macroglobin (A2M), and beta-2 macroglobulin (B2M), or from the group consisting of IL-1, IL-6, IL-7, IL-10, IL-13, IL-15, IL-18, and A2M. The parameters can be cortisol, IL-1, IL-6, IL-7, IL-10, IL-13, IL-18, and A2M; cortisol, IL-1, IL-6, IL-10, IL-13, IL-18, and A2M; IL-1, IL-10, IL-13, IL-18, and A2M; cortisol, IL-1, IL-10, IL-13, IL-18, and A2M; or cortisol, IL-10, IL-13, IL-18, and A2M. Any of the above groups of parameters can further include one or more of neuropeptide Y, ACTH, arginine vasopressin, brain-derived neurotrophic factor, and cortisol. The parameters can further include platelet associated serotonin. The parameters can further include serum or plasma levels of one or more of fatty acid binding protein, alpha-1 antitrypsin, factor VII, epidermal growth factor, glutathione S-transferase, RANTES, plasminogen activator inhibitor type 1, and tissue inhibitor of metalloproteinase type 1.

The numerical values can be biomarker levels in a biological sample from said subject. The biological sample can be whole blood, serum, plasma, urine, or cerebrospinal fluid. The predetermined threshold can be statistical significance (e.g., p<0.05). The subject can be a human.

The method can further comprise providing a numerical value for one or more parameters selected from the group consisting of magnetic resonance imaging, magnetic resonance spectroscopy, computerized tomography scanning, and body mass index. The method can further comprise providing a biological sample from said subject. The method can further comprise measuring said plurality of parameters to obtain said numerical values.

In another aspect, this document features a method for diagnosing a depression disorder in a subject, comprising: (a) providing a biological sample from the subject; (b) measuring a plurality of parameters to obtain numerical values for the parameters, the parameters being predetermined to be relevant to depression; (c) individually weighting each of the numerical values by a predetermined function, each function being specific to each parameter; (d) determining the sum of the weighted values; (e) determining the difference between the sum and a control value; and (f) if the difference is greater than a predetermined threshold, classifying the subject as having depression, or, if the difference is not different than the predetermined threshold, classifying the subject as not having depression. The depression disorder can be MDD.

In another aspect, this document features a method for monitoring treatment for MDD, comprising (a) providing numerical values for a plurality of parameters in a subject diagnosed as having MDD, said parameters being predetermined to be relevant to MDD; (b) using an algorithm comprising said numerical values to calculate an MDD score; (c) repeating steps (a) and (b) after a period of time during which said subject receives treatment for MDD, to obtain a post-treatment MDD score; (d) comparing the post-treatment MDD score from step (c) to the score in step (b) and to a MDD score for normal subjects, and classifying said treatment as being effective if the score from step (c) is closer than the score from step (b) to the MDD score for normal subjects. Step (b) can comprise individually weighting each of said numerical values by a predetermined function, each function being specific to each parameter, and calculating the sum of the weighted values.

The parameters can be selected from the group consisting of IL-1, IL-6, IL-7, IL-10, IL-13, IL-15, IL-18, A2M, and B2M. The period of time can range from weeks to months after the onset of said treatment. A subset of said numerical values can be provided for time points prior to and after initiation of said treatment. The parameters can comprise measurements derived from magnetic resonance imaging, magnetic resonance spectroscopy, or computerized tomography scans. The numerical values can be biomarker levels in a biological sample from said subject. The biological sample can be serum, plasma, urine, or cerebrospinal fluid.

The method can further comprise providing a biological sample from said subject. The method can further comprise measuring the levels of said plurality of parameters to obtain said numerical values.

In another aspect, this document features a method for monitoring treatment for MDD, comprising: (a) providing a biological sample from a subject diagnosed as having MDD; (b) measuring the levels of a plurality of analytes in the sample, the analytes being predetermined to be relevant to MDD; (c) using an algorithm comprising the measured levels to calculate an MDD score; (d) repeating steps (a), (b), and (c) after a period of time during which the subject receives treatment for MDD; (e) comparing the post-treatment MDD score from step (d) to the score in step (c) and to a MDD score for normal subjects, and classifying the treatment as being effective if the score from step (d) is closer than the score from step (c) to the MDD score for normal subjects.

In yet another aspect, this document features a computer-implemented method for diagnosing MDD. The method can include providing a biomarker library database that includes selected biomarker parameters that are predetermined to be relevant to MDD, sets of combinations of the biomarkers and coefficients, the sets of combinations based on clinical data obtained from patients with MDD; and using a computer processor to apply a set of combinations of the biomarkers and associated coefficients to measured values of the biomarkers in the set obtained from a patient based on a predetermined algorithm to produce an MDD score for diagnosing whether the patient has MDD.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF THE DRAWINGS

FIG. 1 is a flow diagram outlining the steps in a method for selection of biomarkers.

FIG. 2 is a flow diagram showing the steps in an exemplary method for developing a disease specific library or panel with an algorithm for diagnostic development.

FIG. 3 is a flow diagram showing steps in a method for developing a basic diagnostic score, where n diagnostic scores are generated.

FIG. 4 is a flow diagram outlining steps in a method for using blood to diagnose, select treatment, monitor treatment efficacy, and optimize therapy.

FIG. 5 shows an example of a computer-based diagnostic system employing the biomarker analysis described in this document.

FIG. 6 shows an example of a computer system that can be used in the computer-based diagnostic system depicted in FIG. 5.

### DETAILED DESCRIPTION

The development of psychotropic drugs has relied on quantification of disease severity through psychopathological parameters (e.g., the Hamilton scale for depression). Subjective factors and lack of a proper definition inevitably influence such parameters. Similarly, diagnostic parameters for enrollment of psychiatric patients in phase II and phase III clinical studies are centered on the assessment of disease severity and specificity by measurement of symptomatological scales, and there are no validated biological correlates for disease trait and state that could help in patient selection. In spite of recent progress in molecular diagnostics, the potential information contained within the patient genotype on the likely phenotypic response to drug treatment has not been effectively captured, particularly in non-research settings.

The techniques described herein are based in part on the identification of methods for establishing a diagnosis of, predisposition to, and prognosis for depression disorder conditions, as well as methods for monitoring treatment of subjects diagnosed with and treated for a depression disorder condition. The methods provided herein can include developing an algorithm, evaluating (e.g., measuring) multiple parameters, and using the algorithm to determine a set of quantitative diagnostic scores. Algorithms incorporating values for multiple biomarkers from biological samples such as serum or plasma can then be applied to patient stratification, and also can be used for identification of pharmacodynamic markers. The approach described herein differs from more traditional approaches to biomarkers in the construction of an algorithm, rather than measuring changes in single markers or groups of single markers at multiple time points.

As used herein, a “biomarker” is a characteristic that can be objectively measured and evaluated as an indicator of a biologic or pathogenic process or a pharmacological response to therapeutic intervention. Biomarkers can be, for example, proteins, nucleic acids, metabolites, physical measurements, or combinations thereof. A “pharmacodynamic” biomarker is a biomarker that can be used to quantitatively evaluate (e.g., measure) the impact of treatment or therapeutic intervention on the course, severity, status, symptomology, or resolution of a disease. As used herein, an “analyte” is a substance or chemical constituent that can be objectively measured and determined in an analytical procedure such as immunoassay or mass spectrometry. An analyte thus can be a type of biomarker.

### Algorithms

Algorithms for determining diagnosis, status, or response to treatment, for example, can be determined for any clinical condition. The algorithms used in the methods provided herein can be mathematic functions incorporating multiple parameters that can be quantified using, without limitation, medical devices, clinical evaluation scores, or biological/chemical/physical tests of biological samples. Each mathematic function can be a weight-adjusted expression of the levels of parameters determined to be relevant to a selected clinical condition. Because of the complexity of the weighting and the multiple marker panels, computers with reasonable computational power typically are required to analyze the data. Algorithms generally can be expressed in the format of Formula 1:

Diagnostic score=f(x1, x2, x3, x4, x5 . . . xn) (1)

The diagnostic score is a value that is the diagnostic or prognostic result, “f” is any mathematical function, “n” is any integer (e.g., an integer from 1 to 10,000), and x1, x2, x3, x4, x5 . . . xn are the “n” parameters that are, for example, measurements determined by medical devices, clinical evaluation scores, and/or tests results for biological samples (e.g., human biological samples such as blood, urine, or cerebrospinal fluid).

The parameters of an algorithm can be individually weighted. An example of such an algorithm is expressed in Formula 2:

Diagnostic score=a1*x1+a2*x2−a3*x3+a4*x4−a5*x5 (2)

Here, x1, x2, x3, x4, and x5 can be measurements determined by medical devices, clinical evaluation scores, and/or test results for biological samples (e.g., human biological samples), and a1, a2, a3, a4, and a5 are weight-adjusted factors for x1, x2, x3, x4, and x5, respectively.

A diagnostic score can be used to quantitatively define a medical condition or disease, or the effect of a medical treatment. For example, an algorithm can be used to determine a diagnostic score for a disorder such as depression. In such an embodiment, the degree of depression can be defined based on Formula 1, with the following general formula:

Depression diagnosis score=f(x1, x2, x3, x4, x5 . . . xn)

The depression diagnosis score is a quantitative number that can be used to measure the status or severity of depression in an individual, “f” is any mathematical function, “n” can be any integer (e.g., an integer from 1 to 10,000), and x1, x2, x3, x4, x5 . . . xn are, for example, the “n” parameters that are measurements determined using medical devices, clinical evaluation scores, and/or test results for biological samples (e.g., human biological samples).

In a more general form, multiple diagnostic scores Sm can be generated by applying multiple formulas to a group of biomarker measurements, as illustrated in equation (3)

Scores Sm=fm(x1, . . . xn) (3)

Multiple scores can be useful for, e.g., sub-indications, such as for diagnosing sub-types of MDD and/or related or unrelated disorders. Some multiple scores also can be parameters indicating patient treatment progress and/or the utility of the treatment selected. For depression disorder, a treatment progress score can help a health care professional (e.g., a doctor or other clinician) adjust treatment doses and duration. A sub-indication score also can help a health care professional to select optimal drugs or combinations of drugs to use for treatment.

### Building Biomarker Libraries

To determine which parameters are useful for inclusion in a diagnostic algorithm, a biomarker library of analytes can be developed, and individual analytes from the library can be evaluated for inclusion in an algorithm for a particular clinical condition. In the initial phases of biomarker library development, the focus may be on broadly relevant clinical content, such as analytes indicative of inflammation, Th1 and Th2 immune responses, adhesion factors, and proteins involved in tissue remodeling (e.g., matrix metalloproteinases (MMPs) and tissue inhibitors of matrix metalloproteinases (TIMPs)). In some embodiments (e.g., during initial library development), a library can include a dozen or more markers, a hundred markers, or several hundred markers. For example, a biomarker library can include a few hundred protein analytes. As a biomarker library is built, new markers can be added (e.g., markers specific to individual disease states, and/or markers that are more generalized, such as growth factors). In some embodiments, analytes can be added to expand the library and to increase specificity beyond the inflammation, oncology, and neuropsychological foci by addition of disease related proteins obtained from discovery research (e.g., using differential display techniques, such as isotope coded affinity tags (ICAT), accurate mass, and time tags). Matrix-assisted laser desorption and ionization (MALDI) and surface enhanced laser desorption/ionization (SELDI) mass spectrometry can provide high-resolution measurements useful for protein biomarker identification and quantification.

The addition of a new analyte to a biomarker library can require a purified or recombinant molecule, as well as the appropriate antibody to capture and detect the new analyte. It is noted that while application of a biomarker library to conventional ELISA platforms can require multiple antibodies for each analyte, a Molecular Interaction Measurement System (MIMS) developed by the Ridge Diagnostics, Inc. (“Ridge;” Research Triangle Park, N.C.; formerly Precision Human Biolaboratories, Inc.) can be operated to use a single specific antibody for each analyte. Although discovery of individual “new or novel” biomarkers is not necessary for developing useful algorithms, such markers can be included. The MIMS platform and other technologies that are suitable for multiple analyte detection methods typically are flexible and open to addition of new analytes. The MIMS platform is a label-free system based on optical sensing and certain features of the MIMI are described in PCT Application No. PCT/US2006/047244 entitled “Optical Molecular Detection” and was published as PCT Publication No. WO 2007/067819, which is incorporated by reference in its entirety as part of the disclosure of this document.

This document provides multiplexed detection systems that can provide robust and reliable measurement of analytes relevant to diagnosing, treating, and monitoring clinical conditions. The biomarker panels can be expanded and transferred to label-free arrays, and algorithms (e.g., computer-based algorithms) can be developed to support clinicians and clinical research.

Custom antibody array(s) can be designed, developed, and analytically validated for about 25-50 antigens. Initially, a panel of about 5 to 10 (e.g., 5, 6, 7, 8, 9, or 10) analytes can be chosen based on their ability to, for example, distinguish affected from unaffected subjects, or to distinguish between stages of disease in patients from a defined sample set. An enriched database, however, usually one in which more than 10 significant analytes are measured, can increase the sensitivity and specificity of test algorithms.

### Selecting Individual Parameters

In the construction of libraries or panels, markers and parameters can be selected using any of a variety of methods. The primary driver for construction of a disease specific library or panel can be knowledge of a parameter's relevance to the disease. To construct a library for diabetes, for example, understanding of the disease would likely warrant the inclusion of blood glucose levels. Literature searches or experimentation also can be used to identify other parameters/markers for inclusion. In the case of diabetes, for example, a literature search might indicate the potential usefulness of hemoglobin A1c (HbAC), while specific knowledge or experimentation might lead to inclusion of the inflammatory markers tumor necrosis factor (TNF)-α receptor 2, interleukin (IL)-6, and C-reactive protein (CRP), which have been shown to be elevated in subjects with type II diabetes.

In some embodiments, parameters that can be used to calculate a depression diagnosis score can include immune system biomarkers. Studies have indicated that inflammation, cytokines, and chemokines may be linked to depression. For example, treatment of patients with cytokines can produce symptoms of depression. Activation of the immune system is observed in many depressed patients, and depression occurs more frequently in those having medical disorders associated with immune dysfunction. Further, activation of the immune system and administration of endotoxin (LPS) or interleukin-1 (IL-1) to animals induces sickness behavior resembling depression, while chronic treatment with antidepressants can inhibit sickness behavior induced by LPS. In addition, several cytokines can activate the hypothalamic-pituitary-adrenal (HPA) axis, which is commonly activated in depressed patients; some cytokines can activate cerebral noradrenergic systems (also commonly observed in depressed patients); and some cytokines/chemokines can activate brain serotonergic systems, which have been implicated in major depressive illness and its treatment.

A wide variety of proteins are involved in inflammation, and any one of them is open to a genetic mutation that impairs or otherwise disrupts the normal expression and function of that protein. Inflammation also induces high systemic levels of acute-phase proteins. These proteins include C-reactive protein, serum amyloid A, serum amyloid P, vasopressin, and glucocorticoids, which cause a range of systemic effects. Inflammation also involves release of proinflammatory cytokines and chemokines.

The immune system has a complex and dynamic relationship with the nervous system, both in health and disease. The immune system surveys the central and peripheral nervous systems, and can be activated in response to foreign proteins, infectious agents, stress, and neoplasia. Conversely, the nervous system modulates immune system function both through the neuroendocrine axis and through vagus nerve efferents. When this dynamic relationship is perturbed, neuropsychiatric diseases can result. In fact, several medical illnesses that are characterized by chronic inflammatory responses (e.g., rheumatoid arthritis) have been reported to be accompanied by depression. In addition, administration of proinflammatory cytokines (e.g., in cancer or hepatitis C therapies) can induce depressive symptomatology. Administration of proinflammatory cytokines in animals induces “sickness behavior,” which is a pattern of behavioral alterations that is very similar to the behavioral symptoms of depression in humans. Thus, the “Inflammatory Response System (IRS) model of depression” (Maes (1999) Adv. Exp. Med. Biol. 461:25-46) proposes that proinflammatory cytokines, acting as neuromodulators, represent key factors in mediation of the behavioral, neuroendocrine and neurochemical features of depressive disorders.

Other classes of biomarkers that may be useful in an algorithm for determining a MDD score include, for example, neurotrophic biomarkers, metabolic biomarkers, and HPA axis biomarkers. The HPA axis (also referred to as the HPTA axis) is a complex set of direct influences and feedback interactions between the hypothalamus (a hollow, funnel-shaped part of the brain), the pituitary gland (a pea-shaped structure located below the hypothalamus), and the adrenal or suprarenal gland (a small, paired, pyramidal organ located at the top of each kidney). The fine, homeostatic interactions between these three organs constitute the HPA axis, which is a major part of the neuroendocrine system that controls reactions to stress and regulates body processes including digestion, the immune system, mood and sexuality, and energy usage.

The following paragraphs provide examples of analytes that can be measured and included in a MDD algorithm, as described further in the Examples herein.

IL-1: IL-1 is strongly involved in the activation of the hypothalamo-pituitary-adrenocortical (HPA) axis. Peripheral and central administration of IL-1 also induces norepinephrine (NE) release in the brain, most markedly in the hypothalamus. Small changes in brain dopamine (DA) are occasionally observed, but these effects are not regionally selective. IL-1 also increases brain concentrations of tryptophan, and the metabolism of serotonin (5-HT) throughout the brain in a regionally nonselective manner. Increases of tryptophan and 5-HT, but not NE, are also elicited by IL-6, which also activates the HPA axis, although it is much less potent in these respects than IL-1.IL-1beta administration to rats stimulated the expression of IL-1beta mRNA in the hypothalamus by 99%, but not that of IL-6. It also significantly activated plasma levels of ACTH, PRL, CORT, and CORT production in adrenal gland. These results indicate that acute peripheral enhancement of IL-1beta may induce neuroendocrine changes also via the immediate activation of its own expression in the hypothalamus, but not that of IL-6 expression in the hypothalamus was found.

IL-6: IL-6 is an interleukin, a pro-inflammatory cytokine. It is secreted by T cells and macrophages to stimulate immune response to trauma, especially burns or other tissue damage leading to inflammation. In addition several studies have indicated that single time measurements of plasma IL-6, revealed significant elevations in depressed patients. IL-6 appears to be involved in the pathogenesis of depression. A study of IL-6-deficient mice (IL-6(−/−)) were subjected to depression-related tests (learned helplessness, forced swimming, tail suspension, sucrose preference). IL-6(−/−) mice showed reduced despair in the forced swim, and tail suspension test, and enhanced hedonic behavior. Moreover, IL-6(−/−) mice exhibited resistance to helplessness. This resistance may be caused by the lack of IL-6, because stress increased IL-6 expression in wild-type hippocampi.

IL-10: Depression is associated with activation of the inflammatory response system. Evidence suggests that pro-inflammatory and anti-inflammatory cytokine imbalance affects the pathophysiology of major depression. Pro-inflammatory cytokines are mainly mediated by T-helper (Th)-1 cells, and include IL-1β, IL-6, TNF-α, and interferon-γ. Anti-inflammatory cytokines are mediated by Th-2 cells, and include IL-4, IL-5, and IL-10. In humans, antidepressants significantly increase production of IL-10.

IL-7: Like IL-10, levels of IL-7 in plasma also were in reduced in depressed male subjects as compared to controls. IL-7 is a hematopoietic cytokine with critical functions in both B- and T-lymphocyte development. IL-7 also exhibits trophic properties in the developing brain. The direct neurotrophic properties of IL-7 combined with the expression of ligand and receptor in developing brain suggest that IL-7 may be a neuronal growth factor of physiological significance during central nervous system ontogeny (Michealson et al. (1996) Dev. Biol. 179:251-263). Adult neurogenesis has been implicated in the etiology and treatment of depression. Elevated stress hormone levels, which are present in some depressed patients and can precipitate the onset of depression, reduce neurogenesis in animal models. Conversely, virtually all antidepressant treatments, including drugs of various classes, electroconvulsive therapy, and behavioral treatments, increase neurogenesis (Drew and Hen (2007) CNS Neurol. Disord. Drug Targets 6:205-218).

IL-13: IL-13 typically acts as an anti-inflammatory cytokine, suggesting that a lower level of IL-13 might increase the dysregulation of the immune system, resulting in increased proinflammatory cytokine activity. Systemic administration of the bacterial endotoxin lipopolysaccharide (LPS) has profound depressive effects on behavior that are mediated by inducible expression of proinflammatory cytokines such as IL-1, IL-6, and tumor necrosis factor-alpha (TNF-alpha) in the brain. When both LPS and IL-13 were co-injected, IL-13 potentiated the depressive effect (Bluthe et al. (2001) Neuroreport 12:3979-3983).

IL-15: IL-15 is a proinflammatory cytokine that is involved in the pathogenesis of inflammatory/autoimmune disease. In addition, IL-15 has been shown to be somatogenic (Kubota et al. (2001) Am. J. Physiol. Regul. Integr. Comp. Physiol. 281:R1004-R1012).

IL-18: Psychological and physical stresses have been reported to exacerbate auto-immune and inflammatory diseases. Plasma concentrations of IL-18 have been shown to be significantly elevated in patients with major depression disorder or panic disorder as compared with normal controls. ACTH stimulates IL-18 expression in human keratinocytes, which provides an insight into the interaction between ACTH and inflammatory mediators. The elevation of plasma IL-18 levels may reflect increased production and release of IL-18 in the central nervous system under stressful settings (see, e.g., Sekiyama (2005) Immunity 22:669-77). Although evaluating IL-18 provided some differentiation of depressed patients from control subjects, this single marker test does not have sufficient diagnostic discrimination power or the robustness to be used in clinical practice.

A2M: A2M is a serum pan-protease inhibitor and an acute phase protein that has been associated with inflammatory disease. A2M also has been implicated in Alzheimer disease based on its ability to mediate the clearance and degradation of A beta, the major component of beta-amyloid deposits. Non-melancholic depressive patients have showed increased A2M serum concentrations in the acute stage of disease and after 2 and 4 weeks of treatment (Kirchner (2001) J. Affect. Disord. 63:93-102).

B2M: B2M is a small (99 amino acid) protein that plays a key role in immunological defense. B2M can be modified by removal of the lysine at position 58, leaving the protein with two disulfide-linked chains of the amino acids 1-57 and 59-99. This modified form (desLys-58-β2-microglobulin, or ΔK58-β2m) has been shown to be associated with chronic inflammatory conditions (Nissen (1993) Danish Med. Bul. 40:56-64). B2M has been found to correlate with disease activity in several autoimmune disorders, and is used as a pharmacodynamic marker of interferon beta treatment in multiple sclerosis.

NPY: NPY is a 36 amino acid peptide neurotransmitter found in the brain and autonomic nervous system. NPY has been associated with a number of physiologic processes in the brain, including the regulation of energy balance, memory and learning, and epilepsy. The main effect of increased NPY is increased food intake and decreased physical activity. A wealth of data indicates that neuropeptides, e.g., NPY, CRH, somatostatin, tachykinins, and CGRP have roles in affective disorders and alcohol use/abuse. Impaired metabolism of plasma NPY and the reduced plasma NPY in patients with MDD may be involved in the pathogenesis or pathophysiology of MDD (Hashamoto et al. (1996) Neurosci. Lett. 216(1):57-60). Thus, as described herein, measuring NYP levels may contribute to the ability to segregate and monitor therapy.

ACTH: ACTH (also referred to as corticotropin) is a polypeptide hormone produced and secreted by the pituitary gland. It is an important player in the hypothalamic-pituitary-adrenal axis. ACTH stimulates the cortex of the adrenal gland and boosts the synthesis of corticosteroids, mainly glucocorticoids but also sex steroids (androgens). Plasma ACTH can be elevated particularly in patients with hypercortisolemia.

AVP: Previous studies have reported abnormalities of neurohypophyseal secretions in major depressive disorder. One of these secretions is AVP, which has been related to MDD in several studies, and particularly in patients with certain subclasses of depression (e.g., melancholic, anxiety-related). Vasopressin, as the name indicates, increases the resistance of the peripheral vessels and thus increases arterial blood pressure. Animal studies have shown that AVP functions as a neuromodulator of the stress response. Human studies have shown that plasma concentrations of AVP increase or decrease under different conditions of stress, whereas normal release is controlled by osmo- and volume receptors. Lastly, plasma levels of AVP were shown to be elevated in patients with MDD (van Londen et al. (1997) Neuropsychopharm. 17:284-292). Measuring AVP levels thus may contribute to the ability to segregate and monitor therapy.

BDNF: BDNF is highly involved in regulation of the HPA axis. In addition, BDNF levels are reduced in depressed patients as compared to controls, and antidepressant treatment can increase serum BDNF levels in depressed patients. The level of plasma BDNF also can be increased with electroconvulsive therapy, suggesting that non-drug therapy can modulate BDNF levels (Marano et al. (2007) J. Clin. Psych. 68:512-7). Univariate analysis (see Example 1 below) identified BDNF as a marker with statistical significance, but the ranges of BDNF levels for the two groups overlap significantly, indicating that serum BDNF by itself is not a good predictor of MDD.

Cortisol: Cortisol is a corticosteroid hormone produced by the adrenal cortex of the adrenal gland. Cortisol is a vital hormone that is often referred to as the “stress hormone,” as it is involved in the response to stress. This hormone increases blood pressure and blood sugar levels, and has an immunosuppressive action. Cortisol inhibits secretion of CRH, resulting in feedback inhibition of ACTH secretion. This normal feedback system may break down when humans are exposed to chronic stress, and may be an underlying cause of depression. Hypercortisolism in depression has been reported, as reflected by elevated mean 24-hour serum cortisol concentrations and increased 24-hour urinary excretion of cortisol. In addition, prolonged hypercortisolemia may be neurotoxic, and recurrent depression episodes associated with elevated cortisol may lead to progressive brain damage.

Questions have been raised regarding evaluation of serum markers for assessing neuropsychiatric diseases. For example, studies investigating testosterone levels and mood disorders have shown conflicting results. More often than not, however, problems with the interpretation of data were due to poor study design. In particular, results from a single assay or a group of assays were considered as single assays rather than analyzed using an algorithm. An expanded library of antibodies (e.g., Ridge's highly multiplexed screening technology, with a capacity of about 200 markers) can be extended to samples (e.g., plasma or serum) from well-characterized patients. In further studies, antibodies for proteins of interest (e.g., monoamines and thyroid hormones) can be used to measure levels in body fluids of patients and controls prior to and during treatment. Surfaces and array designs can be developed to be compatible with samples obtained through a minimally invasive method in order to provide the opportunity for sequential sampling. Sera or plasma typically are used, but, as indicated herein, other biological samples also may be useful. For example, specific monoamines can be measured in urine. In addition, depressed patients as a group have been found to excrete greater amounts of catecholamines and metabolites in urine than healthy control subjects. Analytes of interest include, for example, norepinephrine, epinephrine, vanillylmandelic acid (VMA), and 3-methoxy-4-hydroxyphenylglycol (MHPG). Proteomic studies have indicated that urine is a rich source of proteins and peptides that may be differentially expressed in disease states. Markers associated with neuropsychiatric diseases also can be evaluated (e.g., in collaboration with academic laboratories doing mass spectroscopy-based discovery in cerebrospinal fluid from depressed subjects).

In addition to selected analyte (e.g., protein, peptide, or nucleic acid) markers, algorithms can include other measurable parameters useful in the diagnosis of unipolar depression and/or in distinguishing MDD from other mood disorders (e.g., manic-depressive disorder, post-traumatic stress disorder (PTSD), schizophrenia, seasonal affective disorder (SAD), post-partum depression, and chronic fatigue syndrome). For example, a panel of nine analytes as provided in Table 1 herein, or a sub-set thereof (e.g., as listed in Tables 2-7 herein), either alone or in combination with other measurable parameters, can be used to distinguish MDD from diseases of the elderly that are associated with depression, including, without limitation, vascular dementia, Alzheimer's disease, chronic pain, and disabilities. Similarly, depression in young people seldom presents as a solitary problem but is commonly part of a complex pattern of behavioral concerns, which can be challenging both for diagnosis and treatment. For example, depressed youth often have at least one other concurrent diagnosis, such as anxiety, substance abuse, and disruptive behavior disorders. Further, depressed youth can develop a bipolar mood disorder over time. Diagnosis in such cases can be aided by measuring the levels of specific analytes and calculating a MDD score as described herein.

In some embodiments, a MDD score can include the additional factoring in of other measurable parameters, such as imaging using computerized tomography (CT) scans, magnetic resonance imaging (MRI), molecular resonance spectrography (MRS), other physical measurements such as body mass index (BMI), and measures of thyroid function (e.g., TSH, free thyroxine (fT₄), free triiodothyronine (fT₃), reverse T₃ (rT₃), anti-thyroglobulin antibodies (anti-TG), anti-thyroid peroxidase antibodies (anti-TPO), fT₄/fT₃, and fT₃/rT₃). For example, to sub-classify and further characterize patients, subjects can be imaged with CT scans or MRS, including phosphorus magnetic resonance spectroscopy (³¹P-MRS). Similar studies have suggested that cerebral metabolic changes are implicated in the pathology of MDD. Experiments using ³¹P-MRS have shown that cerebral energy metabolism (e.g., beta-nucleoside triphosphate (beta-NTP), primarily reflecting brain levels of adenosine triphosphate (ATP)), is lower in depressed subjects than in normal controls, and is positively correlated with severity of depression. Beta-NTP levels also appear to correct after successful antidepressant treatment, but not in treatment of non-responders. ³¹P-MRS methods, including 3D chemical shift imaging, provide the possibility to measure ³¹P-MRS metabolites from specific brain regions.

Further, male-female contrasts in estrogen production throughout the reproductive years are proposed to differentially modulate the expression of depression between genders. Mood changes frequently are reported during the late luteal phase of the menstrual cycle and following childbirth. The finding of increased risk for depression at menopause has not been replicated consistently, but a recent epidemiologic study did find that the onset of major depression was increased after menopause, at a time when estrogen levels decline and post-menopausal women are increasingly vulnerable to depression due to this reduced estrogen production. Similarly, while there is a weak relationship between testosterone and depression in general, there is a much stronger relationship between testosterone and depression that does not respond to treatment.

Thus, in some embodiments, the methods described herein can take advantage of the sensitivity and specificity of custom protein arrays for determination of multiple biomarkers from blood, serum, cerebrospinal fluid, and/or urine. In addition, algorithms can reflect concordance between protein signatures and imaging, as well as psychological testing.

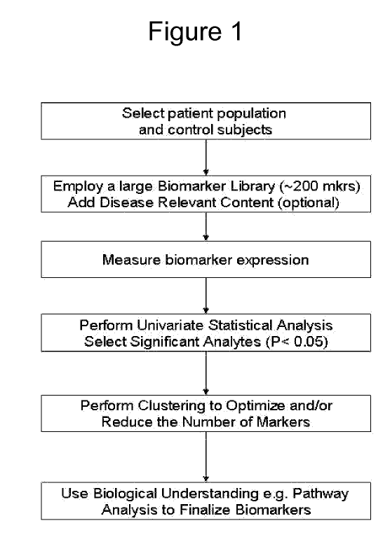

FIG. 1 is a flow diagram detailing the first steps that can be included in development of a disease specific library or panel for use in determining, e.g., diagnosis or prognosis. The process can include two statistical approaches: 1) testing the distribution of biomarkers for association with the disease by univariate analysis; and 2) clustering the biomarkers into groups using a tool that divides the biomarkers into non-overlapping, uni-dimensional clusters, a process similar to principal component analysis. After the initial analysis, a subset of two or more biomarkers from each of the clusters can be identified to design a panel for further analyses. The selection typically is based on the statistical strength of the markers and current biological understanding of the disease.

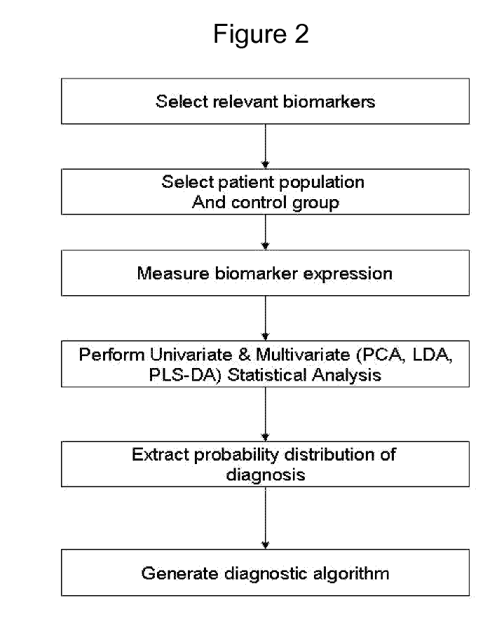

FIG. 2 is a flow diagram depicting steps that can be included to develop a disease specific library or panel for use in establishing diagnosis or prognosis, for example. As shown in FIG. 2, the selection of relevant biomarkers need not be dependant upon the selection process described in FIG. 2, although the first process is efficient and can provide an experimentally and statistically based selection of markers. The process can be initiated, however, by a group of biomarkers selected entirely on the basis of hypothesis and currently available data. The selection of a relevant patient population and appropriately matched (e.g., for age, sex, race, BMI, etc.) population of normal subjects typically is involved in the process. In some embodiments, patient diagnoses can be made using state of the art methodology and, in some cases, by a single group of physicians with relevant experience with the patient population. Biomarker expression levels can be measured using the MIMS instrument or any other suitable technology, including single assays (e.g., ELISA or PCR). Univariate and multivariate analyses can be performed using conventional statistical tools (e.g., not limited to: T-tests, principal components analysis (PCA), linear discriminant analysis (LDA), or Binary Logistic Regression).

### Analyte Measurement and Algorithm Calculation

Methods for diagnosing a depression disorder and monitoring a subject's response to treatment for depression as provided herein can include determining the levels of a group of biomarkers in a biological sample collected from the subject. An exemplary subject is a human, but subjects can also include animals that are used as models of human disease (e.g., mice, rats, rabbits, dogs, and non-human primates). The group of biomarkers can be specific to a particular disease. For example, a plurality of analytes can form a panel specific to MDD.

As used herein, a “biological sample” is a sample that contains cells or cellular material, from which nucleic acids, polypeptides, or other analytes can be obtained. Depending upon the type of analysis being performed, a biological sample can be serum, plasma, or blood cells (e.g., blood cells isolated using standard techniques). Serum and plasma are exemplary biological samples, but other biological samples can be used. Examples of other suitable biological samples include, without limitation, cerebrospinal fluid, pleural fluid, bronchial lavages, sputum, peritoneal fluid, bladder washings, secretions (e.g., breast secretions), oral washings, swabs (e.g., oral swabs), isolated cells, tissue samples, touch preps, and fine-needle aspirates. In some cases, if a biological sample is to be tested immediately, the sample can be maintained at room temperature; otherwise the sample can be refrigerated or frozen (e.g., at −80° C.) prior to assay.

A number of methods can be used to quantify biomarkers (e.g., analytes). For example, measurements can be obtained using one or more medical devices or clinical evaluation scores to assess a subject's condition, or using tests (e.g., biochemical, biophysical, or traditional clinical chemistry analysis) of biological samples to determine the levels of particular analytes. Multiplex methods are particularly useful, as they require smaller sample volumes and perform all of the analysis at one time under the same incubation conditions. An example of platform useful for multiplexing is the FDA approved, flow-based Luminex assay system (xMAP; online at luminexcorp.com). This multiplex technology uses flow cytometry to detect antibody/peptide/oligonucleotide or receptor tagged and labeled microspheres. Since the system is open in architecture, Luminex can be readily adapted to host particular disease panels.

Another useful technique for analyte quantification is immunoassay, a biochemical test that measures the concentration of a substance (e.g., in a biological tissue or fluid such as serum, plasma, cerebral spinal fluid, or urine) based on the specific binding of an antibody to its antigen. Antibodies chosen for biomarker quantification must have a high affinity for their antigens. A vast array of different labels and assay strategies has been developed to meet the requirements of quantifying plasma proteins with sensitivity, accuracy, reliability, and convenience. For example, Enzyme Linked ImmunoSorbant Assay (ELISA) can be used to quantify biomarkers a biological sample. In a “solid phase sandwich ELISA,” an unknown amount of a specific “capture” antibody can be affixed to a surface of a multiwell plate, and the sample can be allowed to absorb to the capture antibody. A second specific, labeled antibody then can be washed over the surface so that it can bind to the antigen. The second antibody is linked to an enzyme, and in the final step a substance is added that can be converted by the enzyme to generate a detectable signal (e.g., a fluorescent signal). For fluorescence ELISA, a plate reader can be used to measure the signal produced when light of the appropriate wavelength is shown upon the sample. The quantification of the assays endpoint involves reading the absorbance of the colored solution in different wells on the multiwell plate. A range of plate readers are available that incorporate a spectrophotometer to allow precise measurement of the colored solution. Some automated systems, such as the BIOMEK® 1000 (Beckman Instruments, Inc.; Fullterton, Calif.), also have built-in detection systems. In general, a computer can be used to fit the unknown data points to experimentally derived concentration curves.

Other techniques that can be used to quantify biomarkers include BIACORE™ Surface Plasmon Resonance (GE Healthcare, Chalfont St. Giles, United Kingdom) and protein arrays. Another instrument that can be used for biomarker quantification without labeling of antigen or antibody is the Molecular Interaction Measurement System (MIMS; Ridge Diagnostics, Inc.). MIMS is nearly reagent free, is rapid, and can be readily used by non-technical individuals.

A number of other higher throughput, multiplexed technologies also can be used to rapidly measure and validate disease-specific and compound-specific biomarkers. These include immunobead based assays, chemoluminescent multiplex assays, and chip and protein arrays. Various protein array substrates can be used, including nylon membranes, plastic microwells, planar glass slides, gel-based arrays, and beads in suspension arrays. In addition to immunoassay-based methodology, high throughput mass spectroscopy-based technologies can be used to both establish the identity and quantify peptides and proteins. The ability of mass spectroscopy to quantify specific protein patterns associated with certain biological conditions within a complex background in an absolute quantitative way can facilitate data standardization, which can be essential for comparing biomarker expression as well as for computational biology and biosimulation.

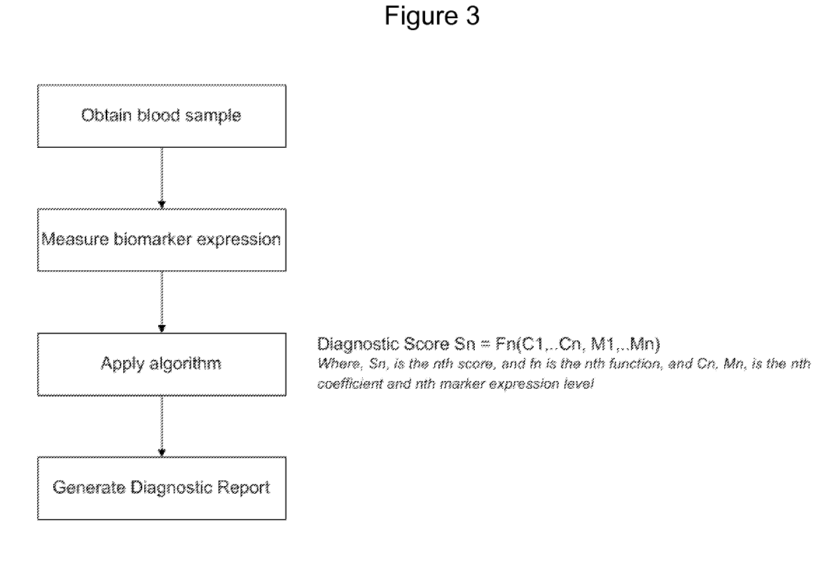

FIG. 3 is a flow diagram depicting steps that can be included in establishing set scores for diagnostic development and application. The process can involve obtaining a biological sample (e.g., a blood sample) from a subject to be tested. Depending upon the type of analysis being performed, serum, plasma, or blood cells can be isolated by standard techniques. If the biological sample is to be tested immediately, the sample can be maintained at room temperature; otherwise the sample can be refrigerated or frozen (e.g., at −80° C.) prior to assay. Biomarker expression levels can be measured using a MIMS instrument or any other suitable technology, including single assays such as ELISA or PCR, for example. Data for each marker are collected, and an algorithm is applied to generate a set diagnostic scores. The diagnostic scores, as well as the individual analyte levels, can be provided to a clinician for use in establishing a diagnosis and/or a treatment action for the subject.

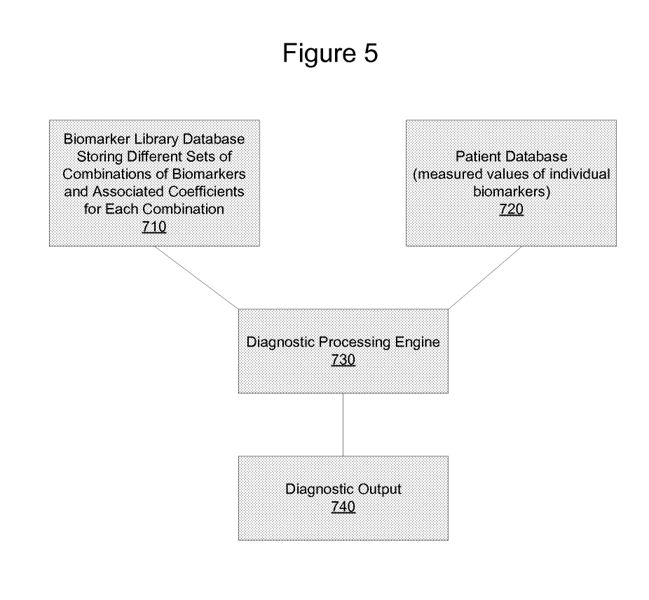

FIG. 5 shows an example of a computer-based diagnostic system employing the biomarker analysis described above. This system includes a biomarker library database 710 that stores different sets combinations of biomarkers and associated coefficients for each combination based on biomarker algorithms which are generated based on, e.g., the method shown in FIG. 1 or 2. The database 710 is stored in a digital storage device in the system. A patient database 720 is provided in this system to store measured values of individual biomarkers of one or more patients under analysis. A diagnostic processing engine 730, which can be implemented by one or more computer processors, is provided to apply one or more sets of combinations of biomarkers in the biomarker library database 710 to the patient data of a particular patient stored in the database 720 to generate diagnostic output for a set of combination of biomarkers that is selected for diagnosing the patient. Two or more such sets may be applied to the patient data to provide two or more different diagnostic output results. The output of the processing engine 730 can be stored in an output device 740, which can be, e.g., a display device, a printer, or a database.

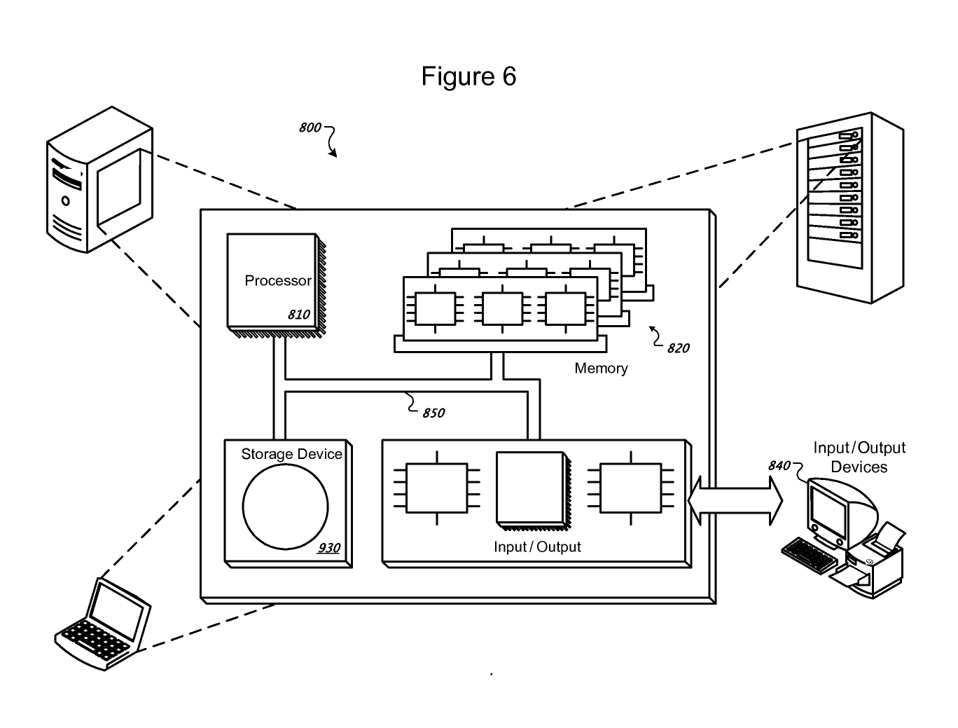

One or more computer systems can be used to implement the system in FIG. 5 and for the operations described in association with any of the computer-implement methods described in this document. FIG. 6 shows an example of such a computer system 800. The system 800 can include various forms of digital computers, such as laptops, desktops, workstations, personal digital assistants, servers, blade servers, mainframes, and other appropriate computers. The system 800 can also include mobile devices, such as personal digital assistants, cellular telephones, smartphones, and other similar computing devices. Additionally the system can include portable storage media, such as, Universal Serial Bus (USB) flash drives. For example, the USB flash drives may store operating systems and other applications. The USB flash drives can include input/output components, such as a wireless transmitter or USB connector that may be inserted into a USB port of another computing device.

In the specific example in FIG. 6, the system 800 includes a processor 810, a memory 820, a storage device 830, and an input/output device 840. Each of the components 810, 820, 830, and 840 are interconnected using a system bus 850. The processor 810 is capable of processing instructions for execution within the system 800. The processor may be designed using any of a number of architectures. For example, the processor 810 may be a CISC (Complex Instruction Set Computers) processor, a RISC (Reduced Instruction Set Computer) processor, or a MISC (Minimal Instruction Set Computer) processor.

In one implementation, the processor 810 is a single-threaded processor. In another implementation, the processor 810 is a multi-threaded processor. The processor 810 is capable of processing instructions stored in the memory 820 or on the storage device 830 to display graphical information for a user interface on the input/output device 840.

The memory 820 stores information within the system 800. In one implementation, the memory 820 is a computer-readable medium. In one implementation, the memory 820 is a volatile memory unit. In another implementation, the memory 820 is a non-volatile memory unit.

The storage device 830 is capable of providing mass storage for the system 800. In one implementation, the storage device 830 is a computer-readable medium. In various different implementations, the storage device 830 may be a floppy disk device, a hard disk device, an optical disk device, or a tape device.

The input/output device 840 provides input/output operations for the system 800. In one implementation, the input/output device 840 includes a keyboard and/or pointing device. In another implementation, the input/output device 840 includes a display unit for displaying graphical user interfaces.

The features described can be implemented in digital electronic circuitry, or in computer hardware, firmware, software, or in combinations of them. The apparatus can be implemented in a computer program product tangibly embodied in an information carrier, e.g., in a machine-readable storage device for execution by a programmable processor; and method steps can be performed by a programmable processor executing a program of instructions to perform functions of the described implementations by operating on input data and generating output. The described features can be implemented advantageously in one or more computer programs that are executable on a programmable system including at least one programmable processor coupled to receive data and instructions from, and to transmit data and instructions to, a data storage system, at least one input device, and at least one output device. A computer program is a set of instructions that can be used, directly or indirectly, in a computer to perform a certain activity or bring about a certain result. A computer program can be written in any form of programming language, including compiled or interpreted languages, and it can be deployed in any form, including as a stand-alone program or as a module, component, subroutine, or other unit suitable for use in a computing environment.

Suitable processors for the execution of a program of instructions include, by way of example, both general and special purpose microprocessors, and the sole processor or one of multiple processors of any kind of computer. Generally, a processor will receive instructions and data from a read-only memory or a random access memory or both. The essential elements of a computer are a processor for executing instructions and one or more memories for storing instructions and data. Generally, a computer will also include, or be operatively coupled to communicate with, one or more mass storage devices for storing data files; such devices include magnetic disks, such as internal hard disks and removable disks; magneto-optical disks; and optical disks. Storage devices suitable for tangibly embodying computer program instructions and data include all forms of non-volatile memory, including by way of example semiconductor memory devices, such as EPROM, EEPROM, and flash memory devices; magnetic disks such as internal hard disks and removable disks; magneto-optical disks; and CD-ROM and DVD-ROM disks. The processor and the memory can be supplemented by, or incorporated in, ASICs (application-specific integrated circuits).

To provide for interaction with a user, the features can be implemented on a computer having a display device such as a CRT (cathode ray tube) or LCD (liquid crystal display) monitor for displaying information to the user and a keyboard and a pointing device such as a mouse or a trackball by which the user can provide input to the computer.

The features can be implemented in a computer system that includes a back-end component, such as a data server, or that includes a middleware component, such as an application server or an Internet server, or that includes a front-end component, such as a client computer having a graphical user interface or an Internet browser, or any combination of them. The components of the system can be connected by any form or medium of digital data communication such as a communication network. Examples of communication networks include a local area network (“LAN”), a wide area network (“WAN”), peer-to-peer networks (having ad-hoc or static members), grid computing infrastructures, and the Internet.

The computer system can include clients and servers. A client and server are generally remote from each other and typically interact through a network, such as the described one. The relationship of client and server arises by virtue of computer programs running on the respective computers and having a client-server relationship to each other.

### Methods for Using Diagnostic Scores

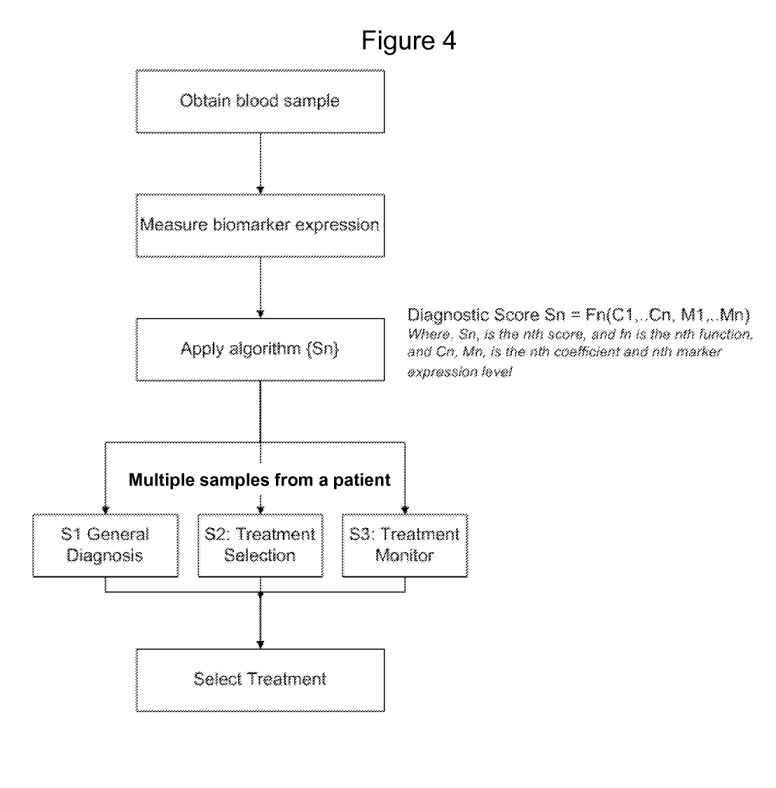

FIG. 4 is a flow diagram illustrating an exemplary process for using diagnostic scores to determine diagnoses, select treatments, and monitor treatment progress. As depicted in FIG. 4, one or more multiple diagnostic scores may be generated using the expression levels of a set of biomarkers. In this example, multiple biomarkers are measured in a subject's blood sample, and three diagnostic scores are generated by the algorithm. In some cases, a single diagnostic score may be sufficient to aid in diagnosis, treatment selection, and monitoring of treatment. When a treatment is selected and treatment begins, the patient still may need to be monitored periodically by measuring biomarker levels (e.g., in a subsequently obtained blood sample) to generate and compare diagnostic scores.

MDD scores can be used to monitor patient status during treatment and to adjust treatment, for example. Nearly half of medical outpatients who receive an antidepressant prescription discontinue treatment during the first month. Patient follow-up and monitoring therefore are extremely important during the first month of treatment. Discontinuation rates within the first three months can reach nearly 70%, depending on the population studied and the agent used (Keller et al. Tnt. Clin. Psychopharmacol. (2002) 17:265-271). Adverse effects of antidepressants are major contributors to treatment failure, as is the perception of lack of efficacy.

Diagnostic scores and/or individual analyte levels or biomarker values can be provided to a clinician for use in establishing or altering a course of treatment for a subject. When a treatment is selected and treatment begins, the subject can be monitored periodically by collecting biological samples at two or more intervals, measuring biomarker levels to generate a diagnostic score corresponding to a given time interval, and comparing diagnostic scores over time. On the basis of these scores and any trends observed with respect to increasing, decreasing, or stabilizing diagnostic scores, a clinician, therapist, or other health-care professional may choose to continue treatment as is, to discontinue treatment, or to adjust the treatment plan with the goal of seeing improvement over time. For example, a change in diagnostic score (e.g., toward a control score for normal individuals not having MDD) can correspond to a positive response to treatment. A change in diagnostic score (e.g., away from a control score for normal individuals not having MDD), or no change in diagnostic score from a baseline level, can indicate failure to respond positively to treatment and/or the need to reevaluate the current treatment plan.

After a patient's diagnostic scores are reported, a health-care professional can take one or more actions that can affect patient care. For example, a health-care professional can record the diagnostic score in a patient's medical record. In some cases, a health-care professional can record a diagnosis of MDD, or otherwise transform the patient's medical record, to reflect the patient's medical condition. In some cases, a health-care professional can review and evaluate a patient's medical record, and can assess multiple treatment strategies for clinical intervention of a patient's condition.

A health-care professional can initiate or modify treatment for MDD symptoms after receiving information regarding a patient's diagnostic score. In some cases, previous reports of diagnostic scores and/or individual analyte levels can be compared with recently communicated diagnostic scores and/or disease states. On the basis of such comparison, a health-care profession may recommend a change in therapy. In some cases, a health-care professional can enroll a patient in a clinical trial for novel therapeutic intervention of MDD symptoms. In some cases, a health-care professional can elect waiting to begin therapy until the patient's symptoms require clinical intervention.

A health-care professional can communicate diagnostic scores and/or individual analyte levels to a patient or a patient's family. In some cases, a health-care professional can provide a patient and/or a patient's family with information regarding MDD, including treatment options, prognosis, and referrals to specialists, e.g., neurologists and/or counselors. In some cases, a health-care professional can provide a copy of a patient's medical records to communicate diagnostic scores and/or disease states to a specialist.

A research professional can apply information regarding a subject's diagnostic scores and/or disease states to advance MDD research. For example, a researcher can compile data on MDD diagnostic scores with information regarding the efficacy of a drug for treatment of MDD symptoms to identify an effective treatment. In some cases, a research professional can obtain a subject's diagnostic scores and/or individual analyte levels to evaluate a subject's enrollment or continued participation in a research study or clinical trial. A research professional can classify the severity of a subject's condition based on the subject's current or previous diagnostic scores. In some cases, a research professional can communicate a subject's diagnostic scores and/or individual analyte levels to a health-care professional, and/or can refer a subject to a health-care professional for clinical assessment of MDD and treatment of MDD symptoms.

Any appropriate method can be used to communicate information to another person (e.g., a professional), and information can be communicated directly or indirectly. For example, a laboratory technician can input diagnostic scores and/or individual analyte levels into a computer-based record. In some cases, information can be communicated by making a physical alteration to medical or research records. For example, a medical professional can make a permanent notation or flag a medical record for communicating a diagnosis to other health-care professionals reviewing the record. Any type of communication can be used (e.g., mail, e-mail, telephone, facsimile and face-to-face interactions). Secure types of communication (e.g., facsimile, mail, and face-to-face interactions) can be particularly useful. Information also can be communicated to a professional by making that information electronically available (e.g., in a secure manner) to the professional. For example, information can be placed on a computer database such that a health-care professional can access the information. In addition, information can be communicated to a hospital, clinic, or research facility serving as an agent for the professional. The Health Insurance Portability and Accountability Act (HIPAA) requires information systems housing patient health information to be protected from intrusion. Thus, information transferred over open networks (e.g., the internet or e-mail) can be encrypted. When closed systems or networks are used, existing access controls can be sufficient.

The following examples provide additional information on various features described above.

### EXAMPLES

### Example 1

### Diagnostic Markers of Depression

Methods provided herein were used to develop a biomarker library and an algorithm for determining depression scores that are useful to diagnose or determine predisposition to MDD, and to evaluate a subject's response to anti-depressive therapeutics. Multiplexed detection systems were used to phenotype molecular correlates of depression. Three statistical approaches were used for biomarker assessment and algorithm development: (1) univariate analysis for testing the distribution of biomarkers for association with MDD; and (2) linear discriminant analysis (LDA) and (3) binary logistic regression for algorithm construction.

Univariate analysis of individual analyte levels: Using the Student's T-test, serum levels of each of the analytes were tested using Luminex multiplex technology and we compared depressed versus normal subjects. The level of significance was set at α≦0.05. Univariate analysis separately explores each variable in a data set. This method looks at the range of values and the central tendency of the values, describes the pattern of response to the variable, and describes each variable on its own. By way of example, FIG. 6 shows the distribution of blood levels for marker X in a hypothetical series of six MDD patients before and after treatment. The first point to be made from this graph is that the concentration of marker X was higher in untreated MDD patients as opposed to control subjects. Second, the levels of marker X in the MDD patients after treatment was similar to that of the control.

The Student's t-Test was then used to compare the two sets of data and to test the hypothesis that a difference in their means is significant. The difference in the means is of statistical significance on the basis of how many standard deviations separate the means. The distance between means is judged significant using Student's t-statistic and its corresponding probability or significance that the absolute value of the t-statistic could be this large or larger by chance. In addition, the t-Test takes into account whether the populations are independent or paired. An independent t-Test can be used when two groups are thought to have the same overall variance but different means. This test can provide support for a statement about how a given population varies from an ideal measure, such as how a treated group compares with an independent control group. The independent t-Test can be performed on data sets with an unequal number of points. In contrast, the paired test is used only when two samples are of equivalent size (i.e., include same number of points). This test assumes that the variance for any point in one population is the same for the equivalent point in the second population. This test can be used to support conclusions about a treatment by comparing experimental results on a sample-by-sample basis. For example, a paired t-Test can be used to compare results for a single group before and after a treatment. This approach can help to evaluate two data sets whose means do not appear to be significantly different using the independent t-Test. During the test(s), the Student's t-Statistic for measuring the significance of the difference between the means is calculated, and the probability (p-Value) that the t-Statistic takes on its value by chance. The smaller the p-Value, the more significant the difference in the means. For many biological systems, an alpha level (or level of significance) of p>0.05 represents the probability that the t-Statistic is achievable just by chance.

For example, application of the students t-Test to the data in FIG. 6 (where there are equal numbers of points in each group) showed that the difference in marker X expression between control subjects and patients with MDD was statistically significant at p>0.002, and the difference in the MDD patients pre- and post-treatment was significant with p>0.013. In contrast, there was no statistically significant difference between the control group and the MDD patients after treatment (p>0.35).

Such data is used to obtain a frequency distribution for the variable. This is achieved by all the values of the variable in order from lowest to highest. The number of appearances for each value of the variable is a count of the frequency with which each value occurs in the data set. By way of example, if a MDD score is calculated using an algorithm as described herein, the patient population can be separated into groups having the same MDD score. If patients are monitored before and after treatment, the frequency for each MDD score can be established, and the effectiveness of the treatment can be ascertained.

PCA and PLS-DA: PCA is mathematically defined as an orthogonal linear transformation that transforms the data to a new coordinate system such that the greatest variance by any projection of the data comes to lie on the first coordinate (called the first principal component), the second greatest variance on the second coordinate, and so on. PCA is used for dimensionality reduction in a data set by retaining those characteristics of the data set that contribute most to its variance, by keeping lower-order principal components and ignoring higher-order ones. Such low-order components often contain the “most important” aspects of the data.

PLS-DA was performed in order to sharpen the separation between groups of observations by rotating PCA components such that a maximum separation among classes was obtained, providing information as to which variables carry the class separating information. PLS-DA and other techniques were used to demonstrate the segregation of normal subjects and depressed patients using the MDD panel to measure serum levels of 16 analytes, all 18 analytes, or sub-sets of four to nine analytes, as examples.

Algorithm based on linear discriminant analysis (LDA): To identify the analytes that contribute the most to discrimination between classes (e.g., depressed vs. normal), a stepwise method of LDA from SPSS 11.0 for Windows was used with following settings: Wilks' lambda (Λ) method was used to select analytes that maximize the cluster separation and analyte entrance into the model was controlled by its F-value. A large F-value indicates that the level of the particular analyte is different between the two groups, and a small F-value (F<1) indicates that there is no difference. In this method, the null hypothesis is rejected for small values of Λ. Thus, the aim was to minimize Λ.

To construct a list of analyte predictors, the F-values for each of the analytes was calculated. Starting with the analyte having the largest F-value (the analyte that differs the most between the two groups), the value of Λ was determined. The analyte with the next largest F-value was then added to the list and Λ was recalculated. If the addition of the second analyte lowered the value of Λ, it was kept in the list of analyte predictors. The process of adding analytes one at a time was repeated until the reduction of Λ no longer occurred.

Cross-validation, a method for testing the robustness of a prediction model, was then carried out. To cross-validate a prediction model, one sample was removed and set aside, the remaining samples were used to build a prediction model based on the pre-selected analyte predictors, and a determination was made as to whether the new model was able to predict the one sample not used in building the new model correctly. This process was repeated for all samples one at a time, and a cumulative cross-validation rate was calculated. The final list of analyte predictors was determined by manually entering and removing analytes to maximize the cross-validation rate, using information obtained from the univariate analyses and cross-validations. The final analyte classifier was then defined as the set of analyte predictors that gives the highest cross-validation rate.

### Example 2

### Choosing Multiple Biomarkers for MDD

Using the Student's t-Test, serum levels of about 100 analytes were tested using Luminex multiplex technology. The data were subsequently analyzed for a comparison of depressed versus normal subjects. The level of significance was set at α≦0.05. After the initial study, the analytes listed in Table 1 were chosen based on statistical significance. This was followed by multivariate analysis (PCA, PLS-DA, LDA) to identify markers that are useful to distinguish MDD patients from normal populations.

Table 1 lists nine biomarkers and indicates the nature of the potential relationship of each analyte to the pathophysiology of depression disorder. In practical use, a smaller group of biomarkers may be sufficient to aid in diagnosis and treatment monitoring for MDD, either with or without additional information derived from a clinical evaluation. Several others examples using different marker sets were established and are shown in Tables 2-7. MDD algorithms with sub-sets of four to nine analytes have demonstrated diagnostic sensitivity in the range of 70% to 90%. These groups, or combinations of these groups with other information, also are used to distinguish different subtypes of unipolar depression, stratify patients, and/or to select and monitor treatments.

TABLE 1

| ANALYTE | RELATIONSHIP TO DEPRESSION |
|---|---|
| IL-1 | strongly involved with activation of the HPA axis in MDD |
| IL-13 | usually acts as an anti-inflammatory cytokine |
| IL-7 | may be a neuronal growth factor |
| IL-6 | plasma IL-6 is elevated in MDD |
| IL-18 | stress related release of IL-18 in CNS and plasma |
| A2M | associated with inflammatory disease and depression |
| IL-15 | a novel proinflammatory cytokine |
| IL-10 | usually acts as an anti-inflammatory cytokine |
| B2M | can be associated with chronic inflammatory conditions |

TABLE 2
Complete nine member inflammatory marker centric depression panel
ANALYTE
IL-1
IL-13
IL-7
IL-6
IL-18
A2M
IL-15
IL-10
B2M

TABLE 3
Representative eight member inflammatory marker centric depression panel
ANALYTE
IL-1
IL-13
IL-7
IL-6
IL-18
A2M
IL-15
IL-10

TABLE 4
Representative seven member inflammatory marker centric depression panel
ANALYTE
IL-1
IL-13
IL-7
IL-6
IL-18
A2M
IL-10

TABLE 5
Representative six member inflammatory marker centric depression panel
ANALYTE
IL-1
IL-13
IL-6
IL-18
A2M
IL-10

TABLE 6
Representative five member inflammatory marker centric depression panel
ANALYTE
IL-1
IL-13
IL-18
A2M
IL-10

TABLE 7
Representative four member inflammatory marker centric depression panel
ANALYTE
IL-13
IL-18
A2M
IL-10

The potential relevance of each marker in Tables 1-7 to MDD is discussed in further detail herein.

### Example 3

### Use of an Algorithm to Calculate MDD Scores and Assess Treatment

Using all nine of the markers listed in Table 1, as well as NPY, a diagnostic score was established based on the following algorithm:

Depression diagnosis score=f(a1*IL-1+a2*IL-13+a3*IL-7+a4*IL-6+a5*IL-18+a6*A2M+a7*IL-15+a8*IL-10+a9*B2M+a10*NPY

Using five of the markers listed above (A2M, IL-1, IL-10, IL-13, and IL-18), a diagnostic score was established based on the following algorithm:

Depression diagnosis score=f(a1*A2M+a2*IL-1+a3*IL-10+a4*IL-13+a5*IL-18).

Several other examples of depression algorithms using different marker sets were established and are shown in Tables 3-7. MDD algorithms with subsets of four to six analytes have shown diagnostic sensitivity in the range of 70% to 90%. The analyte lists shown in Tables 3-7 represent sub-sets of immune-related biomarkers for depression. These panels are not meant to be the only possible combinations of marker that would be useful; they do, however, represent panels that should provide statistically valid adjuncts to diagnosis and monitoring patients with depression.

While this document contains many specifics, these should not be construed as limitations on the scope of an invention or of what may be claimed, but rather as descriptions of features specific to particular embodiments of the invention. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or a variation of a subcombination.

Only a few embodiments are disclosed. Variations and enhancements of the described embodiments and other embodiments can be made based on what is described and illustrated in this document.

1. A method for characterizing depression in a subject, comprising
(a) providing a numerical value for a plurality of analytes relevant to depression;
(b) individually weighting each numerical value in a manner specific to each analyte to obtain a weighted value for each analyte;
(c) determining a result value based on an equation that includes each weighted value;
(d) determining the difference between said result value and a control result value, wherein the control result value was determined in a manner comparable to that of the result value, with the proviso that each control numerical value corresponds to the level of said analyte in a biological sample from a normal subject; and
(e) if said difference is greater than a threshold indicative of depression, classifying said subject as having depression, or, if said difference is not greater than said threshold, classifying said subject as not having depression.

2. The method of claim 1, wherein said depression is associated with major depressive disorder (MDD).

3. The method of claim 1, wherein said analytes are selected from the group consisting of interleukin-1 (IL-1), interleukin-6 (IL-6), interleukin-7 (IL-7), interleukin-10 (IL-10), interleukin-13 (IL-13), interleukin-15 (IL-15), interleukin-18 (IL-18), alpha-2-macroglobin (A2M), and beta-2 macroglobulin (B2M).

4. The method of claim 1, wherein said analytes are selected from the group consisting of cortisol, IL-1, IL-6, IL-7, IL-10, IL-13, IL-15, IL-18, and A2M.

5-9. (canceled)

10. The method of claim 4, wherein said analytes further comprise one or more of neuropeptide Y, ACTH, arginine vasopressin, and brain-derived neurotrophic factor.

11. The method of claim 4, wherein said analytes further comprise platelet associated serotonin.

12. The method of any claim 4, wherein said analytes further comprise serum or plasma levels of one or more of fatty acid binding protein, alpha-1 antitrypsin, factor VII, epidermal growth factor, glutathione S-transferase, RANTES, plasminogen activator inhibitor type 1, and tissue inhibitor of metalloproteinase type 1.

13. The method of claim 1, wherein said numerical values are analyte levels in a biological sample from said subject.

14. The method of claim 13, wherein said biological sample is whole blood, serum, plasma, urine, or cerebrospinal fluid.

15-18. (canceled)

19. The method of claim 1, wherein said result value is statistically different from said control result value at p<0.05.

20. (canceled)

21. The method of claim 1, wherein said subject is a human.

22. The method of claim 1, further comprising providing a numerical value for one or more parameters selected from the group consisting of magnetic resonance imaging, magnetic resonance spectroscopy, computerized tomography scanning, and body mass index.

23. The method of claim 1, further comprising providing a biological sample from said subject.

24. The method of claim 1, further comprising measuring the level of each of said plurality of analytes to obtain said numerical values.

25. A method for monitoring treatment for MDD, comprising:
(a) providing a numerical value for a plurality of parameters in a subject diagnosed as having MDD, said parameters being relevant to MDD;
(b) using an algorithm comprising said numerical values to calculate an MDD score;
(c) repeating steps (a) and (b) after a period of time during which said subject receives treatment for MDD, to obtain a post-treatment MDD score;
(d) comparing the post-treatment MDD score from step (c) to the score in step (b) and to a MDD score for normal subjects, and classifying said treatment as being effective if the score from step (c) is closer than the score from step (b) to the MDD score for normal subjects.

26. The method of claim 25, wherein step (b) comprises individually weighting each numerical value in a manner specific to each parameter to obtain a weighted value for each parameter, and calculating a result value based on an equation that includes each weighted value.

27. The method of claim 25, wherein said parameters comprise analytes selected from the group consisting of IL-1, IL-6, IL-7, IL-10, IL-13, IL-15, IL-18, A2M, and B2M.

28. The method of claim 25, wherein said period of time ranges from weeks to months after the onset of said treatment.

29. The process of claim 25, wherein a subset of said numerical values are provided for time points prior to and after initiation of said treatment.

30. The method of claim 25, wherein said parameters comprise measurements derived from magnetic resonance imaging, magnetic resonance spectroscopy, or computerized tomography scans.

31. The method of claim 25, wherein said numerical values are biomarker levels in a biological sample from said subject.

32. The method of claim 31, wherein said biological sample is serum, plasma, urine, or cerebrospinal fluid.

33-35. (canceled)

36. The method of claim 25, further comprising providing a biological sample from said subject.

37. The method of claim 25, further comprising measuring the levels of said plurality of parameters to obtain said numerical values.

38. A computer-implemented method for diagnosing MDD, comprising:
providing a biomarker library database that includes selected biomarker parameters that are relevant to MDD, sets of combinations of the biomarkers, and coefficients the sets of combinations based on clinical data obtained from patients with MDD; and
using a computer processor to apply a set of combinations of the biomarkers and associated coefficients to measured values of the biomarkers in the set obtained from a patient based on an algorithm to produce an MDD score for diagnosing whether the patient has MDD.
